(19) United States
(12) Patent Application Publication
(10) Pub. No.: US 2007/0134191 A1
(43) Pub. Date: Jun. 14, 2007

(54) Hair styling compositions containing a nonionic amphiphilic polymer in combination with styling polymers and emulsifying agents

(75) Inventor: Jim M. Singer, South Orange, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/302,664

(22) Filed: Dec. 14, 2005

(51) Int. Cl. A61K 8/73

(52) U.S. Cl. 424/70.17; 424/70.7

(57) ABSTRACT

A process for treating a keratinous substrate involving contacting the keratinous substrate with a composition containing: (a) at least one nonionic amphiphilic polymer having at least one fatty chain and at least one hydrophilic unit; (b) at least one fixative polymer; (c) at least one emulsifying agent; and (d) a cosmetically acceptable medium.

### BACKGROUND OF THE INVENTION

The present invention generally relates to a composition and process for treating hair based on a hair styling composition that facilitates the shaping and reshaping of hair and provides various levels of hold to hair.

The more common hair styling compositions on the cosmetic market for shaping and/or maintaining the hairstyle are spray compositions containing a solution, usually alcohol- or water-based, and one or more materials, generally polymer resins. One of the functions of polymer resins is to form links between the hairs, these materials also being called fixatives, in a mixture with various cosmetic adjuvants. This solution is generally packaged either in an appropriate aerosol container, which is pressurized with the aid of a propellant, or in a pump flask.

Other known hair styling compositions include styling gels and mousses, which are generally applied to the wetted hair before brushing or setting it. In contrast to the conventional aerosol lacquers, these compositions have the disadvantage that they do not allow the hair to be fixed in a shape created before their application. In fact, these compositions are essentially aqueous and their application wets the hair and is therefore unable to maintain the initial shape of the hairstyle. In order to shape and fix the hairstyle, therefore, it is necessary to carry out subsequent brushing and/or drying.

Such hair styling compositions all have the same disadvantage that they do not allow the hairstyle to be later modified to a desired shape, which is other than that formed initially, without starting the styling and fixing operations again. Moreover, under various kinds of stress, the hairstyle has a tendency to take on an undesirable permanent set, which cannot easily be modified. Also in the styling process, one desires hair conditioning benefits, such as ease of combing and soft hair feel appearance.

Fixing the hairstyle is an important element in hair styling, and involves maintaining a shaping that has already been carried out, or simultaneously shaping and fixing the hair. Thus, hair styling compositions that facilitate the shaping and reshaping of hair are highly desirable. In addition, hair styling compositions that provide various levels of hold to hair present consumers with a range of hair styling products to choose from.

In accordance with the invention, the term “hair styling composition” relates to any kind of hair composition that can be used to effect hair styling, for example, fixing compositions, setting compositions, permanent waving compositions, and hair treatment products.

### BRIEF SUMMARY OF THE INVENTION

It has surprisingly been found that hair styling compositions having a unique texture may be formulated with the use of a certain type of nonionic amphiphilic polymer, in the absence of betaines.

Thus, the present invention relates to a cosmetic composition for styling hair containing:

at least one nonionic amphiphilic polymer having at least one fatty chain and at least one hydrophilic unit;

at least one fixative polymer;

at least one emulsifying agent; and

a cosmetically acceptable medium.

The present invention is also directed to a process for shaping and reshaping hair involving contacting the hair with the above-disclosed cosmetic composition.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

The cosmetic compositions and methods of the present invention can comprise, consist of, or consist essentially of the essential elements and limitations of the invention described herein, as well as any additional or optional ingredients, components, or limitations described herein or otherwise useful in personal care compositions intended for topical application to the hair.

According to one embodiment of the present invention, there is provided a cosmetic composition for styling a keratinous substrate such as hair. The cosmetic composition is based on a combination of at least one nonionic amphiphilic polymer having at least one fatty chain and at least one hydrophilic unit, at least one fixative polymer, at least one emulsifying agent, and a cosmetically acceptable medium.

It has been surprisingly found that a hair styling composition having a unique texture and is capable of imparting numerous styling benefits when applied onto the hair, can be formulated in the absence of a betaine.

Nonionic Amphiphilic Polymer

The nonionic amphiphilic polymers having at least one fatty chain and at least one hydrophilic unit, which are used according to the invention, are preferably chosen from:

(1) celluloses modified with groups containing at least one fatty chain such as, for example:

hydroxyethyl celluloses modified with groups containing at least one fatty chain such as alkyl, arylalkyl or alkylaryl groups or mixtures thereof, and in which the alkyl groups are preferably C₈-C₂₂, such as the product Natrosol Plus Grade 330 CS(C₁₆ alkyls) sold by the company Aqualon, or the product Bermocoll EHM 100 sold by the company Berol Nobel, those modified with polyalkylene glycol alkylphenyl ether groups, such as the product Amercell Polymer HM-1500 (polyethylene glycol (15) nonylphenyl ether) sold by the company Amerchol.

(2) hydroxypropyl guars modified with groups containing at least one fatty chain, such as the product Esaflor HM 22 (C₂₂ alkyl chain) sold by the company Lamberti, and the products Miracare XC₉₅-3 (C₁₄ alkyl chain) and RE205-1 (C₂₀ alkyl chain) sold by the company Rhone-Poulenc.

(3) polyether urethanes containing at least one fatty chain such as a C₈-C₃₀ alkyl or alkenyl group, for instance the products Dapral T210 and Dapral T212, now known respectively as Elfacos T210 and Elfacos T212, sold by the company Akzo Nobel.

The nonionic amphiphilic polymer having at least one fatty chain and at least one hydrophilic unit is present in the composition in an amount of from 0.01% to 20% by weight, preferably in an amount of from 0.05% to 15% by weight, and more preferably in an amount of from 0.1% to 10%, by weight, based on the weight of the composition. Particularly preferred nonionic amphiphilic polymers having at least one fatty chain and at least one hydrophilic unit are PPG-5 palmeth-60 hexyl dicarbamate, commercially available from Akzo Nobel under the tradename Elfacos T-212, and PPG-14 laureth-60 hexyl dicarbamate, commercially available from Akzo Nobel under the tradename Elfacos T-210.

Fixative Polymers

The fixative polymers used in the present invention which may be chosen from anionic, cationic, amphoteric and non-ionic polymers and mixtures thereof. The fixative polymer may additionally be halogenated, in particular fluorinated.

The fixative polymers can be used in solubilized form or else in the form of dispersions of solid polymer particles (latex or pseudo-latex).

The cationic fixative polymers which can be used according to the present invention can be selected from polymers containing primary, secondary, tertiary and/or quaternary amine groups forming part of the polymer chain or linked directly to it and having a molecular weight of between 500 and approximately 5,000,000 and preferably between 1000 and 3,000,000.

The anionic fixative polymers that are generally used are polymers containing groups derived from carboxylic, sulphonic or phosphoric acid and have a weight-average molecular weight of between approximately 500 and 5,000,000.

The carboxylic groups are provided by unsaturated mono- or dicarboxylic acids monomers such as those corresponding to the formula (II): 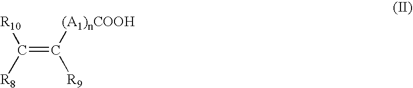
in which:
n is an integer from 0 to 10,
A₁ denotes a methylene group, optionally connected to the carbon atom of the unsaturated group, or to the neighboring methylene group when n is greater than 1, via a hetero atom such as oxygen or sulphur,
R₁₀ denotes a hydrogen atom or a phenyl or benzyl group,
R₈ denotes a hydrogen atom or a lower alkyl or carboxyl group, and
R₉ denotes a hydrogen atom, a lower alkyl group or a —CH₂—COOH, phenyl or benzyl group.

In the abovementioned formula, a lower alkyl radical preferably denotes a group having 1 to 4 carbon atoms and in particular methyl and ethyl.

The anionic fixative polymers containing carboxylic groups which can be used according to the invention are:

A) Homo- or copolymers of acrylic or methacrylic acid or salts thereof and in particular the products sold under the names VERSICOL E or K by the company Allied Colloid and ULTRAHOLD by the company BASF. The copolymers of acrylic acid and of acrylamide sold in the form of their sodium salt under the names RETEN 421, 423 or 425 by the company Hercules, the sodium salts of polyhydroxycarboxylic acids.

B) Copolymers of acrylic or methacrylic acids with a monoethylenic monomer such as ethylene, styrene, vinyl esters, acrylic acid esters or methacrylic acid esters. These copolymers can be grafted onto a polyalkylene glycol such as polyethylene glycol and optionally crosslinked. Mention may be made in particular of the copolymers containing an optionally N-alkylated and/or hydroxyalkylated acrylamide unit in their chain sold under the name QUADRAMER by the company American Cyanamid. Mention may also be made of copolymers of acrylic acid and of C₁-C₄ alkyl methacrylate and terpolymers of vinylpyrrolidone, of (meth)acrylic acid and of (meth)acrylate of C₁-C₂₀ alkyl, for example lauryl (such as the product sold by the company ISP under the name ACRYLIDONE LM), tert-butyl (LUVIFLEX VBM 70 sold by BASF) or methyl (STEPANHOLD EXTRA sold by Stepan) and methacrylic acid/ethyl acrylate/tert-butyl acrylate terpolymers such as the product sold under the name LUVIMER 100 P by the company BASF.

C) Copolymers derived from crotonic acid such as those containing vinyl acetate or propionate units in their chain and optionally other monomers such as allylic esters or methallylic esters, vinyl ether or vinyl ester of a linear or branched saturated carboxylic acid with a long hydrocarbon chain such as those containing at least 5 carbon atoms, it being possible for these polymers optionally to be grafted and crosslinked, or alternatively a vinyl, allylic or methallylic ester of an α- or β-cyclic carboxylic acid. Commercial products falling into this class are the resins 28-29-30, 26-13-14 and 28-13-10 sold by the company National Starch.

D) Copolymers derived from C₄-C₈ monounsaturated carboxylic acids or anhydrides selected from:

copolymers comprising:

(i) one or more maleic, fumaric or itaconic acids or anhydrides and

(ii) at least one monomer selected from vinyl esters, vinyl ethers, vinyl halides, phenylvinyl derivatives, acrylic acid and its esters, the anhydride functions of these copolymers optionally being monoesterified or monoamidated. Commercially available examples thereof include, but are not limited to, GANTREZ AN or ES and AVANTAGE CP and Aquaflex FX-64 from the company ISP;

copolymers comprising:

(i) one or more maleic, citraconic or itaconic anhydrides and

(ii) one or more monomers selected from allylic or methallylic esters optionally containing one or more acrylamide, methacrylamide, α-olefin, acrylic or methacrylic ester, acrylic or methacrylic acid or vinylpyrrolidone groups in their chain, the anhydride functions of these copolymers optionally being monoesterified or monoamidated.

E) Polyacrylamides containing carboxylate groups.

The polymers comprising sulphonic groups are polymers containing vinylsulphonic, styrenesulphonic, naphthalenesulphonic or acrylamidoalkylsulphonic units.

These polymers can be selected in particular from:

polyvinylsulphonic acid salts having a weight-average molecular weight ranging from approximately 1000 to approximately 100,000, as well as the copolymers with an unsaturated comonomer such as acrylic or methacrylic acids and their esters, as well as acrylamide or its derivatives, vinyl ethers and vinylpyrrolidone; polystyrenesulphonic acid salts, the sodium salts having a weight-average molecular weight of about 500,000 and about 100,000, which are sold respectively under the names FLEXAN 500 and FLEXAN 130 by National Starch. One example thereof is polyacrylamidoethylpropanesulphonic acid sold under the name COSMEDIA POLYMER HSP 1180 by Henkel.

Anionic fixative polymers may be also be sulphonated polyesters comprising repeating units representable by the following general formula:

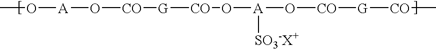

in which A and G represent divalent radicals and X represents an alkali metal, especially sodium or potassium. Among the preferred sulphonated polyesters according to the invention, A represents an arylene radical, especially phenylene, and G represents a linear or branched alkylene radical optionally interrupted by one or more oxygen atoms, or a cycloalkylene radical. When G represents a linear or branched alkylene radical optionally interrupted by one or more oxygen atoms, the radical —O-G-O— is preferably a (poly)alkylene glycol residue containing 1 to 20 alkylene glycol units. The alkylene radical is preferably, according to the invention, a lower, linear or branched C₂-C₄ alkylene radical, more preferably an ethylene radical Among these polymers, preference will be given to those marketed under the names AQ 1045, AQ 1350 and AQ 14000 by the company EASTMAN CHEMICAL, more particularly AQ 1350.

According to the invention the anionic fixative polymers are selected from acrylic acid copolymers such as the acrylic acid/ethyl acrylate/N-tert-butylacrylamide terpolymers sold in particular under the name ULTRAHOLD STRONG by the company BASF, copolymers derived from crotonic acid such as the vinyl acetate/vinyl tert-butylbenzoate/crotonic acid terpolymers and the crotonic acid/vinyl acetate/vinyl neododecanoate terpolymers sold in particular under the name RESIN 28-29-30 by the company National Starch, polymers derived from maleic, fumaric or itaconic acids or anhydrides with vinyl esters, vinyl ethers, vinyl halides, phenylvinyl derivatives or acrylic acid and its esters, such as the methyl vinyl ether/maleic anhydride monoesterified copolymers sold, for example, under the name GANTREZ by the company ISP, the copolymers of methacrylic acid and of methyl methacrylate sold under the name EUDRAGIT L by the company Rohm Pharma, the copolymers of methacrylic acid/methyl methacrylate/C₁-C₄ alkyl acrylate/acrylic acid or C₁-C₄ hydroxyalkyl methacrylate which are sold in the form of dispersions under the name AMERHOLD DR 25 by the company Amerchol or under the name ACUDYNE 255 by the company Rohm & Haas, the copolymers of methacrylic acid and of ethyl acrylate sold under the name LUVIMER MAEX or MAE by the company BASF and the vinyl acetate/crotonic acid copolymers and the vinyl acetate/crotonic acid copolymers grafted with polyethylene glycol, sold under the name ARISTOFLEX A by the company BASF.

The anionic fixative polymers which are more preferred are selected from the monoesterified methyl vinyl ether/maleic anhydride copolymers sold under the name GANTREZ ES 425 by the company ISP, the acrylic acid/ethyl acrylate/N-tert-butylacrylamide terpolymers sold under the name ULTRAHOLD STRONG by the company BASF, the copolymers of methacrylic acid and of methyl methacrylate sold under the name EUDRAGIT L by the company Rohm Pharma, the vinyl acetate/vinyl tert-butylbenzoate/crotonic acid terpolymers and the crotonic acid/vinyl acetate/vinyl neododecanoate terpolymers sold under the name RESIN 28-29-30 by the company National Starch, the copolymers of methacrylic acid and of ethyl acrylate sold under the name LUVIMER MAEX or MAE by the company BASF and the vinyl pyrrolidone/acrylic acid/lauryl methacrylate terpolymers sold under the name ACRYLIDONE LM by the company ISP.

The amphoteric fixative polymers which can be used in accordance with the invention can be selected from polymers containing units B and C distributed randomly in the polymer chain, in which B denotes a unit derived from a monomer containing at least one basic nitrogen atom and C denotes a unit derived from an acid monomer containing one or more carboxylic or sulphonic groups, or alternatively B and C can denote groups derived from carboxybetaine or sulphobetaine zwitterionic monomers; B and C can also denote a cationic polymer chain containing primary, secondary, tertiary or quaternary amine groups, in which at least one of the amine groups bears a carboxylic or sulphonic group connected via a hydrocarbon radical or alternatively B and C form part of a chain of a polymer containing an α,β-dicarboxylic ethylene unit in which one of the carboxylic groups has been made to react with a polyamine containing one or more primary or secondary amine groups.

The amphoteric fixative polymers corresponding to the definition given above which are more particularly preferred are selected from the following polymers:

(1) Polymers resulting from the copolymerization of a monomer derived from a vinyl compound bearing a carboxylic group such as, more particularly, acrylic acid, methacrylic acid, maleic acid, α-chloroacrylic acid, and a basic monomer derived from a substituted vinyl compound containing at least one basic atom, such as, more particularly, dialkylaminoalkyl methacrylate and acrylate, dialkylaminoalkylmethacrylamides and -acrylamides.

(2) Polymers containing units derived from:

a) at least one monomer selected from acrylamides and methacrylamides substituted on the nitrogen with an alkyl radical,

b) at least one acidic comonomer containing one or more reactive carboxylic groups, and

c) at least one basic comonomer such as esters containing primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides which are more particularly preferred according to the invention are groups in which the alkyl radicals contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are selected more particularly from acrylic acid, methacrylic acid, crotonic acid, itaconic acid, maleic acid and fumaric acid and alkyl monoesters, having from 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose CTFA name is octylacrylamide/acrylates/butylaminoethyl methacrylate copolymer such as the products sold under the name AMPHOMER or LOVOCRYL 47 by the company National Starch are particularly used.

(3) crosslinked and alkylated polyamino amides partially or totally derived from polyamino amides of formula (III): [symbol]
CO—R₁₀—CO-Z [symbol] (III)
in which:
R₁₀ represents a divalent radical derived from a saturated dicarboxylic acid, a mono- or dicarboxylic aliphatic acid containing an ethylenic double bond, an ester of a lower alkanol, having from 1 to 6 carbon atoms, of these acids or a radical derived from the addition of any one of the acids to a bis(primary) or bis(secondary) amine, and
Z denotes a bis(primary), mono- or bis(secondary) polyalkylene-polyamine radical and preferably represents:
a) in proportions of from 60 to 100 mol %, the radical
—NH [symbol] (CH₂)ₓ—NH [symbol] ₚ (IV)
where x=2 and p=2 or 3, or alternatively x=3 and p=2 this radical being derived from diethylenetriamine, from triethylenetetraamine or from dipropylenetriamine;
b) in proportions of from 0 to 40 mol %, the radical (IV) above in which x=2 and p=1 and which is derived from ethylenediamine, or the radical derived from piperazine: 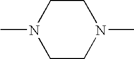
c) in proportions of from 0 to 20 mol %, the —NH—(CH₂)₆—NH— radical derived from hexamethylenediamine, these polyamino amines being crosslinked by addition of a difunctional crosslinking agent selected from epihalohydrines, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids are preferably selected from acids having from 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The alkane sultones used in the alkylation are preferably propane sultone or butane sultone, the salts of the alkylating agents are preferably the sodium or potassium salts.

(4) polymers containing zwitterionic units of formula (V): 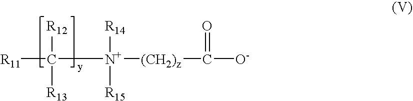
in which:
R₁₁ denotes a polymerizable unsaturated group such as an acrylate, methacrylate, acrylamide or methacrylamide group,
y and z represent an integer ranging from 1 to 3,
R₁₂ and R₁₃ represent a hydrogen atom, methyl, ethyl or propyl,
R₁₄ and R₁₅ represent a hydrogen atom or an alkyl radical such that the sum of the carbon atoms in R₁₄ and R₁₅ does not exceed 10.

The polymers comprising such units can also contain units derived from non-zwitterionic monomers such as dimethyl or diethylaminoethyl acrylate or methacrylate or alkyl acrylates or methacrylates, acrylamides or methacrylamides or vinyl acetate.

By way of example, mention may be made of the copolymer of methyl methacrylate/dimethyl carboxymethylammonio methyl ethylmethacrylate such as the product sold under the name DIAFORMER Z301 by the company Sandoz.

(5) Polymers derived from chitosan containing monomer units corresponding to the following formulae: 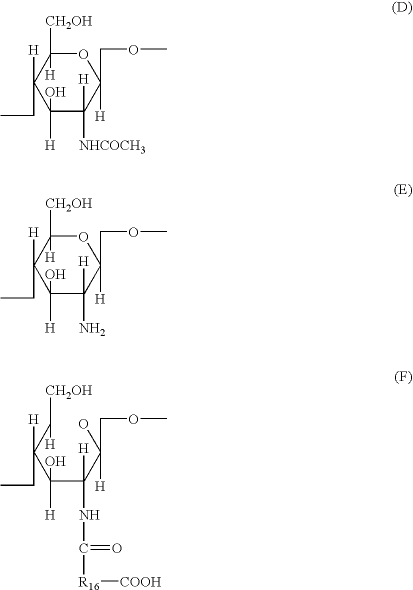
the unit D being present in a concentration ranging from 0 to 30%,
the unit E in a concentration ranging from 5 to 50%, and
the unit F in a concentration ranging from 30 to 90%,
it being understood that, in this unit F, R₁₆ represents a radical of formula: 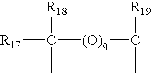
in which,
if q=0, R₁₇, R₁₈ and R₁₉ each independently represent a hydrogen atom, a methyl, hydroxyl, acetoxy or amino residue, a monoalkylamine residue or a dialkylamine residue which are optionally interrupted by one or more nitrogen atoms and/or optionally substituted with one or more amine, hydroxyl, carboxyl, alkylthio or sulphonic groups, an alkylthio residue in which the alkyl group bears an amino residue, at least one of the radicals R₁₇, R₁₈ and R₁₉ being, in this case, a hydrogen atom;
or, if q=1, R₁₇, R₁₈ and R₁₉ each independently represent a hydrogen atom, as well as the salts formed by these compounds with bases or acids.

(6) Polymers derived from the N-carboxyalkylation of chitosan, such as N-carboxymethylchitosan or N-carboxybutylchitosan sold under the name “EVALSAN” by the company Jan Dekker.

(7) Polymers corresponding to formula (VI): 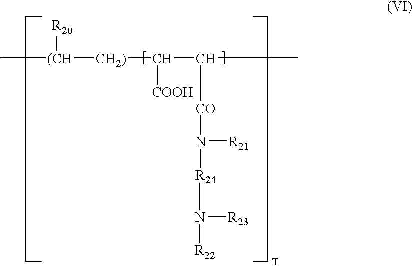
in which:
R₂₀ represents a hydrogen atom, a CH₃O, CH₃CH₂O or phenyl radical,
R₂₁ denotes hydrogen or a lower alkyl radical such as methyl or ethyl,
R₂₂ denotes hydrogen or a lower alkyl radical such as methyl or ethyl,
R₂₃ denotes a lower alkyl radical such as methyl or ethyl or a radical corresponding to the formula: —R₂₄—N(R₂₂)₂, wherein
R₂₂ can be the same or different,
R₂₄ representing a —CH₂—CH₂—, —CH₂—CH₂—CH₂— or —CH₂—CH(CH₃)— group, and the higher homologues of these radicals containing up to 6 carbon atoms.

(8) Amphoteric polymers of the type -D-X-D-X selected from:

a) polymers obtained by the action of chloroacetic acid or sodium chloroacetate on compounds containing at least one unit of formula:
=D=X=D=X=D= (VII)
where D denotes a radical 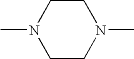
and X denotes the symbol E or E′, E or E′, which may be identical or different, denotes a divalent radical which is an alkylene radical containing a straight or branched chain containing up to 7 carbon atoms in the main chain, which is unsubstituted or substituted with hydroxyl groups and which can contain, in addition to the oxygen, nitrogen and sulphur atoms, from 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups.
b) Polymers of formula:
-D-X-D-X- (VII)
in which D denotes a radical 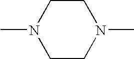
and X denotes the symbol E or E′ and at least once E′, E having the meaning given above and E′ is a divalent radical which is an alkylene radical with a straight or branched chain having up to 7 carbon atoms in the main chain, which is unsubstituted or substituted with one or more hydroxyl radicals and containing one or more nitrogen atoms, the nitrogen atom being substituted with an alkyl chain which is optionally interrupted by an oxygen atom and necessarily containing one or more carboxyl functions or one or more hydroxyl functions and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also contain other vinyl comonomers such as vinylcaprolactam.

The amphoteric fixative polymers which are particularly preferred according to the invention are those of family (3), such as the copolymers whose CTFA name is octylacrylamide/acrylates/butylaminoethyl methacrylate copolymer, such as the products sold under the names AMPHOMER, AMPHOMER LV 71 or LOVOCRYL 47 by the company National Starch and those of family (4) such as the copolymer of methyl methacrylate/dimethyl carboxymethylammonio methyl ethylmethacrylate, sold, for example, under the name DIAFORMER Z301 by the company Sandoz.

The anionic or amphoteric fixative polymers can, if necessary, be partially or totally neutralized. The neutralizing agents are, for example, sodium hydroxide, potassium hydroxide, 2-amino-2-methyl-1-propanol, monoethanolamine, triethanolamine or triisopropanolamine and inorganic or organic acids such as hydrochloric acid or citric acid.

The non-ionic fixative polymers useful according to the present invention are, in particular, polyurethanes.

The fixative polymer is present in the composition in an amount of from 0.01% to 25% by weight, preferably in an amount of from 0.05% to 20% by weight, and more preferably in an amount of from 0.1% to 15%, by weight, based on the weight of the composition. Particularly preferred styling polymers are polyvinylpyrrolidone, vinylpyrrolidone/vinyl acetate copolymer, AMP-acrylates/allyl methacrylate copolymer (commercially available from Noveon under the tradename, Fixate G-100), sodium polystyrene sulfonate (commercially available from National Starch under the tradename, Flexan II), Vinylpyrrolidone/acrylates/lauryl methacyrlate copolymer (commercially available from ISP under the tradename, Acrylidone LM), polyquaternium-6, and polyurethane-2 (commercially available from Noveon under the tradename, Avalure 405 or 410).

Emulsifying Agents

The emulsifying agents which can be used according to the invention are those having an HLB of less than 7 and in particular fatty acid esters of polyols such as mono-, di-, tri- or sesqui-oleates or -stearates of sorbitol or glycerol, laurates of glycerol or polethylene glycol; alkyl or alkoxy dimethicone copolyols having an alkyl or alkoxy chain pendent or at the end of a silicone-based backbone having for example from 6 to 22 carbon atoms. The emulsifying agents may also be those having an HLB greater than 7 such as fatty acid esters of polyethylene glycol (monostearate or monolaurate of polyethylene glycol); esters of fatty acids (stearate, oleate) of sorbitol which are polyoxyethylenated; polyoxy ethylenated alkyl (lauryl, cetyl, stearyl, octyl) ethers and dimethicone copolyols. In general, it is possible to use nonionic or anionic or cationic emulsifiers well known to persons skilled in the art.

The nonionic type emulsifiers are fatty acids or amides of polyalkoxylated and/or polyglycerolated fatty acids; polyoxyethylenated and/or polyoxypropylenated fatty alcohols (i.e., compounds prepared by reacting an aliphatic fatty alcohol such as behenyl or cetyl alcohol with ethylene oxide or propylene oxide or an ethylene oxide/propylene oxide mixture); fatty acid esters of polyols, optionally polyoxyethylenated and/or polyoxypropylenated (i.e., compounds prepared by reacting a fatty acid such as stearic acid or oleic acid with a polyol such as, for example, an alkylene glycol or glycerol or a polyglycerol, optionally in the presence of ethylene oxide or propylene oxide or an ethylene oxide/propylene oxide mixture); and polyalkoxylated and/or polyglycerolated alkylphenols; or polyalkoxylated and/or polyglycerolated 1,2- or 1,3-alkanediols; and alkylethers of polyalkoxylated and/or polyglycerolated 1,2- or 1,3-alkanediols or alkenediols, or mixtures thereof.

The polyalkoxylated fatty acids are commercial products, and are principally products sold under the mark “Myrj”.

The esters of fatty acids and polyoxyethylenated polyols for which the polyol is sorbitol are known products (Polysorbate and products sold under the mark “Tween”).

The polyoxyethylenated fatty alcohols are commercial products and principally those sold under the mark “Brij”.

The fatty acids or amides of polyglycerolated fatty acids are also commercial products such as those sold under the mark “Plurol” (Gattefosse) or “Drewpol” (Stefan Company).

The emulsifying agents according to the invention can also be anionic surfactants which may have a hydrophilic-lipophilic balance (HLB) ranging from 10 to 40. They are principally salts of fatty acids (for example alkaline salts or organic salts such as amine salts), the said fatty acids having, for example, from 12 to 18 carbon atoms and being able to have a double bond as in the case of oleic acid; the alkaline salts or salts of organic bases of alkyl-sulfuric and alkyl-sulfonic acids having 12 to 18 carbon atoms, of alkyl-arylsulfonic acids whose alkyl chain contains 6 to 16 carbon atoms, the aryl group being, for example, a phenyl group. They are also ether-sulfates, in particular, the sulfatation products of fatty alcohols and polyalkoxylated alkylphenols, in which the aliphatic chain has from 6 to 20 carbon atoms and the polyalkoxylated chain has from 1 to 30 oxyalkylene units, in particular oxyethylene, oxypropylene or oxybutylene. All these anionic surfactants are well known and many among them are commercial products.

The emulsifying agents according to the invention can also be cationic surfactants such as quaternary ammonium derivatives.

The emulsifying agent is present in the composition in an amount of from 0.05% to 15% by weight, preferably in an amount of from 0.1% to 10% by weight, and more preferably in an amount of from 0.5% to 6.0% by weight, based on the weight of the composition. Particularly preferred emulsifying agents are Isoceteth-20, Polysorbate 20, PEG-40 hydrogenated castor oil, oleth-2, cetyl alcohol and glyceryl stearate.

Cosmetically Acceptable Medium

The cosmetically acceptable medium of the composition is preferably an aqueous medium consisting of water and may advantageously comprise at least one cosmetically acceptable organic solvent such as, for example, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or ethers thereof such as, for example, propylene glycol monomethyl ether, butylene glycol, dipropylene glycol and also diethylene glycol alkyl ethers, for instance diethylene glycol monoethyl ether or monobutyl ether, or alternatively polyols, for instance glycerol. Polyethylene glycols, polypropylene glycols and mixtures of all these compounds may also be used as the solvent.

The solvents may then be present in the composition in an amount of from 10% to 95% by weight, preferably from 20% to 90% by weight, and more preferably from 60% to 85% by weight, based on the weight of the composition.

Optional Ingredients/Additives

The styling composition of the present invention can also comprise hair-conditioning agents. Conditioning agents can be selected from oily substances, silicones, non-ionic substances, cationic amphiphilic ingredients, cationic polymers or their mixtures. Oily substances are selected from compounds such as silicone oils, either volatile or non-volatile, natural and synthetic oils. Silicone oils that can be used include dimethicone, dimethiconol, polydimethylsiloxane, DC fluid ranges from Dow Corning, natural oils such as olive oil, almond oil, avocado oil, weizenkeim oil, ricinus oil and the synthetic oils, such as mineral oil.

Silicones that can be used as conditioning agents are dimethicone, cyclopentasiloxane, cyclohexasiloxane, amodimethicone, silicone emulsions, and dimethicone copolyols.

Non-ionic conditioning agents can be polyols such as glycerin, glycol and derivatives, polyethyleneglycols known with trade names Carbowax PEG from Union Carbide and Polyox WSR range from Amerchol, polyglycerin, polyethyleneglycol mono or di fatty acid esters. Non-ionic conditioning agents can also be esters and hydrocarbons such as caprylic/capric triglycerides, isopropyl palmitate, isododecane, polyisobutene.

Cationic amphiphilic conditioning agents ingredients can also be used. Typical examples of those ingredients are cetyl trimethly ammonium chloride, stear trimonium chloride, dipalmitoyl dimonium chloride, distearyl dimethyl ammonium chloride, stearamidopropyl trimonuim chloride, dioleoylethyl dimethyl ammonium methosulfate, dioleoylethyl hydroxyethylmonium methosulfate. Those can at the same time serve as solubilizing agents for those ingredients difficult to integrate into the formulations.

The cationic polymers mentioned above used for achieving styling are as well found to be suitable for hair conditioning purposes when formulated into the compositions of the present invention.

Typical concentration range for any of the conditioners mentioned above can be from 0.01% to 5% by weight, preferably from 0.01% to 3.5% by weight, and more preferably from 0.05% to 2.5% by weight, based on the weight of the composition.

The composition(s) of the present invention may also comprise additives, for instance those chosen from the non-exhaustive list such as reducing agents, antioxidants, sequestering agents, softeners, antifoams, moisturizers, emollients, basifying agents, gelling agents, wetting agents, thickening agents, spreading agents, dispersants, plasticizers, sunscreens, direct dyes or oxidation dyes, pigments, mineral fillers, clays, colloidal minerals, nacres, nacreous agents, fragrances, peptizers, preserving agents, fixing or non-fixing polymers, ceramides, proteins, antioxidants, active agents, vitamins, antidandruff agents, aliphatic or aromatic alcohols, and more particularly ethanol, benzyl alcohol, modified or unmodified polyols, such as glycerol, glycol, propylene glycol, dipropylene glycol, butylene glycol or butyl diglycol, volatile silicones, mineral, organic or plant oils, oxyethylenated or non-oxyethylenated waxes, paraffins, fatty acids, associative or non-associative thickening polymers, fatty amides, fatty esters, fatty alcohols, and the like.

According to another embodiment of the present invention, there is also provided a process for treating a keratinous substrate, such as hair, involving contacting the substrate with a composition containing: at least one nonionic amphiphilic polymer having at least one fatty chain and at least one hydrophilic unit; at least one fixative polymer; at least one emulsifying agent; and a cosmetically acceptable medium. The process allows for the styling of substrates, such as hair, in a cosmetically appealing manner and in a variety of desired styles. Moreover, previously shaped hair can be re-shaped without the need for re-application of additional product. Various levels of hold can be obtained by applying the composition onto dry, damp or towel-dried hair. The composition may also be used on all types of curly hair to give natural suppleness but still maintain good curl definition.

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

### EXAMPLES

### Example 1—Hair Styling Gel

| CHEMICAL OR INCI NAME | % |
|---|---|
| AMP-ACRYLATES/ALLYL METHACRYLATE | 3.9987 |
| COPOLYMER | |
| PPG-14 PALMETH-60 HEXYL DICARBAMATE | 4 |
| POLYETHYLENE GLYCOL | 0.24 |
| PEG-40 HYDROGENATED CASTOR OIL | 0.75 |
| DIMETHICONE | 2 |
| BEESWAX | 2 |
| LAURETH-4 | 0.116 |
| LAURETH-23 | 0.084 |
| PVP | 1.94 |
| ISOCETETH-20 | 4 |
| PRESERVATIVES, FRAGRANCES, OTHER ADDITIVES | 5.311 |
| WATER | q.s. to |
| | 100% |

### Example 2—Hair Styling Gel

| CHEMICAL OR INCI NAME | % |
|---|---|
| AMP-ACRYLATES/ALLYL METHACRYLATE | 3.78 |
| COPOLYMER | |
| BUTYLENE GLYCOL | 1 |
| MICA | 0.0375 |
| PPG-14 PALMETH-60 HEXYL DICARBAMATE | 3.5 |
| OLETH-10 | 0.01 |
| NYLON-12 | 0.01 |
| POLYETHYLENE GLYCOL | 0.288 |
| PEG-90M | 0.1999 |
| GLYCERIN | 1 |
| PEG-40 HYDROGENATED CASTOR OIL | 0.9 |
| CALCIUM SODIUM BOROSILICATE | 0.02405 |
| PVP | 3 |
| ISOCETETH-20 | 4 |
| PRESERVATIVES, FRAGRANCES, OTHER ADDITIVES | 0.731 |
| WATER | q.s. to |
| | 100% |

### Example 3—Hair Styling Cream

| CHEMICAL OR INCI NAME | % |
|---|---|
| AMP-ACRYLATES/ALLYL METHACRYLATE | 2.9997 |
| COPOLYMER | |
| BUTYLENE GLYCOL | 1 |
| MICA | 0.202 |
| KAOLIN | 0.5 |
| PPG-14 PALMETH-60 HEXYL DICARBAMATE | 3 |
| NYLON-12 | 0.01 |
| POLYETHYLENE GLYCOL | 0.24 |
| PEG-90M | 0.1999 |
| PEG-40 HYDROGENATED CASTOR OIL | 0.75 |
| DIMETHICONE | 2 |
| BEESWAX | 2 |
| LAURETH-4 | 0.116 |
| LAURETH-23 | 0.084 |
| PVP | 2 |
| ISOCETETH-20 | 4 |
| PPG-5-CETETH-20 | 3 |
| PRESERVATIVES, FRAGRANCES, OTHER ADDITIVES | 3.136 |
| WATER | q.s. to |
| | 100% |

### Example 4—Hair Styling Paste

| CHEMICAL OR INCI NAME | % |
|---|---|
| AMP-ACRYLATES/ALLYL METHACRYLATE | 3.9987 |
| COPOLYMER | |
| BUTYLENE GLYCOL | 3.5 |
| TRIBEHENIN | 2 |
| PPG-14 PALMETH-60 HEXYL DICARBAMATE | 4 |
| POLYETHYLENE GLYCOL | 0.24 |
| PEG-90M | 0.1999 |
| HYDROXYPROPYL GUAR | 0.01 |
| PEG-40 HYDROGENATED CASTOR OIL | 0.75 |
| DIMETHICONE | 2 |
| CETYL ESTERS | 2 |
| CARAMEL | 0.03 |
| LAURETH-4 | 0.116 |
| LAURETH-23 | 0.084 |
| PVP | 3 |
| ISOCETETH-20 | 4 |
| PPG-5-CETETH-20 | 3 |
| PRESERVATIVES, FRAGRANCES, OTHER ADDITIVES | 1.811 |
| WATER | q.s. to |
| | 100% |

An evaluation test on 12 panelists was performed in order to assess the performance of the inventive styling composition of Example 5 below versus a control styling composition that did not contain the nonionic amphiphilic polymer having at least one fatty chain and at least one hydrophilic unit, after their respective application onto hair.

### Example 5—Hair Styling Gel

| CHEMICAL OR INCI NAME | % |
|---|---|
| PVP | 3 |
| AMP-ACRYLATES/ALLYL METHACRYLATE | 14 |
| COPOLYMER | |
| BUTYLENE GLYCOL | 1 |
| PPG-14 PALMETH-60 HEXYL DICARBAMATE | 3.5 |
| GLYCERIN | 1 |
| PEG-40 HYDROGENATED CASTOR OIL (and) | 1.2 |
| POLYETHYLENE GLYCOL | |
| ISOCETETH-20 | 4 |
| PEG-90M | 0.2 |
| OLETH-10 | 0.01 |
| PRESERVATIVES, FRAGRANCES, OTHER ADDITIVES | 0.919 |
| WATER | q.s. to |
| | 100% |

It was observed that the inventive styling composition had statistically significantly higher scores (i.e., better performance) than the control styling composition in terms of the following attributes on the hair:

ease of distribution;

slip and wetting properties;

wet combing properties;

dry combing properties;

smooth feel on wet hair;

supple feel on wet hair;

ease of passing the fingers through the hair;

ease of blow drying;

less clumping of composition on the hair;

ability to separate strands;

dry combing properties.

Thus, it can be seen that the composition in accordance with the present invention imparts better styling properties to hair than a styling composition that does not contain a nonionic amphiphilic polymer having at least one fatty chain and at least one hydrophilic unit.

What is claimed is:

1. A process for treating a keratinous substrate comprising contacting the keratinous substrate with a composition containing:
(a) at least one nonionic amphiphilic polymer having at least one fatty chain and at least one hydrophilic unit;
(b) at least one fixative polymer;
(c) at least one emulsifying agent; and
(d) a cosmetically acceptable medium.

2. The process of claim 1 wherein the at least one nonionic amphiphilic polymer is present in an amount of from about 0.1% to about 10.0% by weight, based on the weight of the composition.

3. The process of claim 1 wherein the at least one nonionic amphiphilic polymer is present in an amount of from about 0.5% to about 8.0% by weight, based on the weight of the composition.

4. The process of claim 1 wherein the at least one nonionic amphiphilic polymer is present in an amount of from about 1.0% to about 5.0% by weight, based on the weight of the composition.

5. The process of claim 1 wherein the at least one nonionic amphiphilic polymer is a polyether urethane having at least one fatty chain.

7. The process of claim 1 wherein the at least one nonionic amphiphilic polymer is PPG-14 palmeth-60 hexyl dicarbamate.

8. The process of claim 1 wherein the at least one nonionic amphiphilic polymer is PPG-14 laureth-60 hexyl dicarbamate.

9. The process of claim 1 wherein the at least one fixative polymer is present in an amount of from about 0.05% to about 25.0% by weight, based on the weight of the composition.

10. The process of claim 1 wherein the at least one fixative polymer is present in an amount of from about 0.1% to about 15.0% by weight, based on the weight of the composition.

11. The process of claim 1 wherein the at least one fixative polymer is selected from an anionic fixative polymer, a nonionic fixative polymer, a cationic fixative polymer, and an amphoteric fixative polymer.

12. The process of claim 1 wherein the at least one emulsifying agent is present in an amount of from about 0.05% to about 15.0% by weight, based on the weight of the composition.

13. The process of claim 1 wherein the at least one emulsifying agent is present in an amount of from about 0.5% to about 6.0% by weight, based on the weight of the composition.

14. The process of claim 1 wherein the at least one emulsifying agent is selected from a nonionic emulsifier, an anionic emulsifier and a cationic emulsifier.

15. A composition used for treating a keratinous substrate comprising:
(a) at least one nonionic amphiphilic polymer having at least one fatty chain and at least one hydrophilic unit;
(c) at least one styling polymer;
(d) at least one emulsifying agent; and
(e) a cosmetically acceptable medium.

16. The composition of claim 15 wherein the at least one nonionic amphiphilic polymer is present in an amount of from about 0.1% to about 10.0% by weight, based on the weight of the composition.

17. The composition of claim 15 wherein the at least one nonionic amphiphilic polymer is present in an amount of from about 0.5% to about 8.0% by weight, based on the weight of the composition.

18. The composition of claim 15 wherein the at least one nonionic amphiphilic polymer is present in an amount of from about 1.0% to about 5.0% by weight, based on the weight of the composition.

19. The composition of claim 15 wherein the at least one nonionic amphiphilic polymer is a polyether urethane having at least one fatty chain.

20. The composition of claim 15 wherein the at least one nonionic amphiphilic polymer is PPG-14 palmeth-60 hexyl dicarbamate.

21. The composition of claim 15 wherein the at least one nonionic amphiphilic polymer is PPG-14 laureth-60 hexyl dicarbamate.

22. The composition of claim 15 wherein the at least one fixative polymer is present in an amount of from about 0.05% to about 25.0% by weight, based on the weight of the composition.

23. The composition of claim 15 wherein the at least one fixative polymer is present in an amount of from about 0.1% to about 15.0% by weight, based on the weight of the composition.

24. The composition of claim 15 wherein the at least one fixative polymer is selected from an anionic fixative polymer, a nonionic fixative polymer, a cationic fixative polymer, and an amphoteric fixative polymer.

25. The composition of claim 15 wherein the at least one emulsifying agent is present in an amount of from about 0.05% to about 15.0% by weight, based on the weight of the composition.

26. The composition of claim 15 wherein the at least one emulsifying agent is present in an amount of from about 0.5% to about 6.0% by weight, based on the weight of the composition.

27. The composition of claim 15 wherein the at least one emulsifying agent is selected from a nonionic emulsifier, an anionic emulsifier and a cationic emulsifier.

28. The composition of claim 15 wherein the composition is free of a betaine.
